(19) United States
(12) Patent Application Publication
(10) Pub. No.: US 2004/0096419 A1
(43) Pub. Date: May 20, 2004

(54) Cosmetic preparation containing vitamin a

(76) Inventors: Karin Golz-Berner, Monaco (MC); Leonhard Zastrow, Monaco (MC)

Correspondence Address: Stephen A. Pendorf, Pendorf & Cutliff, 5111 Memorial Highway, Tampa, FL, 33634-7356, US

(21) Appl. No.: 10/474,093

(22) PCT Filed: Mar. 27, 2002
(86) PCT No.: PCT/DE02/01147

(30) Foreign Application Priority Data
Apr. 4, 2001 (DE) 10117842.5

(51) Int. Cl. A61K 7/06; A61K 9/127; A61K 35/78; A61K 31/22

(52) U.S. Cl. 424/74; 424/450; 424/735; 424/769; 514/549

(57) ABSTRACT

The invention relates to a preparation containing vitamin A and the use thereof as a cosmetic. The aim of the invention is to visibly improve the cosmetic effect of retinol or retinoides on the skin. According to the invention, the preparation comprises a retinyl derivative, a retinyl derivative encapsulated in 150-800 nm liposomes, 40-100 nm retinol microcapsules and a cherry extract, acerola, pineapple and/or bark extract from white qeubracho/silkworm extracts or a mixture thereof. The extract contains retinol or a retinyl derivative in additional to cosmetic auxiliary active ingredients, carrier active ingredients and/or other active ingredients.

### FIELD OF THE INVENTION

The invention relates to a preparation containing vitamin A and the use thereof as a cosmetic.

### DESCRIPTION OF THE RELATED ART

The use of vitamin A and of its derivatives in cosmetics has been known for a long time. Also in this context, a number of publications are known where retinol or retinoides were combined with other components in order to enhance the effectiveness and stability of these compounds. EP-0666735 B1 describes the use of a skin whitener in which retinols are combined with different substances, such as ascorbic acid, niacin, kojic acid, liquorice extract and others. A combination of retinoides with vitamin B3 compounds is known from WO 00/24371. WO 00/27352 describes topical compositions with a collagen-enhancing effect which contain a wheat protein, a retinoid, a vitamin E derivative and ascorbic acid.

### SUMMARY OF THE INVENTION

The aim of the invention is to visibly improve the cosmetic effect of retinol and/or retinoides on the skin, in particular to prolong the same.

This task is solved according to the invention by a combination of different forms of application of retinol or of its derivatives together with a specific plant extract.

Therefore, the cosmetic preparation according to the invention, which contains vitamin A, comprises

a) 0.001 to 1.0 percent by weight of a retinyl derivative, selected from among retinyl palmitate, retinyl ascorbate and mixtures thereof, related to the weight of the cosmetic preparation;

b) 0.01 to 10 percent by weight of a retinyl derivative, selected from among retinyl palmitate, retinyl ascorbate and mixtures thereof and encapsulated in phospholipid-containing liposomes with a particle size of the liposomes in the range of 150 to 800 nm, the content of retinyl derivatives being related to the weight of the liposomes;

c) 0.01 to 5 percent by weight of micro-capsules with pure retinol the particle size of the capsules being in the range of 40 to 100 nm and which contain pure retinol in the range of 0.5 to 3 percent by weight, related to the weight of the filled capsules;

d) 0.1 to 20 percent by weight, related to the weight of the cosmetic preparation, of an extract selected from among the aqueous fruit extracts cherry extract (Prunus cerasus), acerola extract (Malpighia punicifolia), pineapple extract, hydrolized bark extract of Quebracho blanco, silkworm extract and mixtures of these extracts, with the content of the extract of at least one retinol compound, selected from among retinol, retinyl palmitate, retinyl ascorbate and mixtures thereof, being in the range of 0.01 to 5 percent by weight, this figure being related to the dry weight of the extract; and

e) the rest, up to 100 percent by weight, of cosmetic auxiliaries, carrier substances and mixtures thereof.

A cherry extract of particular preference is the aqueous extract of the Spanish cherry.

The cherry extract contains preferably retinyl palmitate in a mixture together with at least one other vitamin, selected from among a vitamin E derivative, vitamin C, vitamin F and mixtures thereof, and carotenoids. The extract is preferably encapsulated in capsules with a diameter of 30-100 μm.

The plant extracts are preferably contained with 3-20%, in particular 5-20 percent by weight.

Carotenoids within the meaning of the invention are, for instance, β-carotene, flavoxanthine, neurosporin, lycopin.

Vitamin E derivatives within the meaning of the invention are, for instance, tocopheryl acetate, tocopheryl palmitate.

Another embodiment of the invention comprises the extract which preferably being an extract mixture of the Spanish cherry (Prunus cerasus) and of the acerola fruit (Malpighia punicifolia).

Surprisingly, it was found that by combining the different free and encapsulated forms of retinol/retinoid together with certain plant extracts which contain retinol and/or other vitamins and their precursors, a longer bonding of the retinol to the skin surface and/or in the upper skin layers takes place than when they are applied individually and thus a visibly enhanced effectiveness with the same or similar concentrations of common retinol contents can be achieved.

The stimulation of the cell transformation of the epidermis and the differentiation of the newly formed keratinocytes are, in particular, regarded as the effect of the retinol derivatives. By this, a visible anti-wrinkle effect is achieved as well as a protection against scavengers which in the present combination is maintained over a surprisingly long period of time.

The liposomes that contain a retinyl derivative are completely closed lipid-bilayer membranes on the basis of phospholipides which retain an aqeous volume enclosed.

Liposomes may be unilamellar vesicles (having a single membrane bilayer) or multilamellar vesicles (onion-like structures characterised by multi-membrane bilayers each of which are separated from the next one by an aqueous layer). The bilayer consists of two lipid monolayers having a hydrophobic “tail” area and a hydrophilic “head” area. The structure of the membrane bilayer is such that the hydrophobic (homopolar) “tails” of the lipid monolayer orient themselves in the direction of the centre of the bilayer while the hydrophilic “heads” orient themselves in the direction of the aqueous phase.

The making of liposomes, from saturated and unsaturated lipides, has been described in a very great number of patents, as well as their use as a carrier system. The retinyl derivative may be incorporated in the usual manner.

The following may, for instance, be used as phospholipides: phosphatidylcholine, phosphatidylethanolamine, phosphatidyl-inositol, phosphatidylserin, phosphatide acid and lysolecithine as well as mixtures thereof. Known products are, for instance, Phoslipon® or NAT®.

The retinol-containing microcapsules consist preferably of a shell made of a carboxymethylcellulose-chitosan complex. This shell of a biopolymer stabilizes the retinol, which is suspended in an aqueous or aqueous oily medium, against spontaneous decomposition reactions. Special additives for preservation/stabilization and for emulsification may be included, such as parabens, cellulose rubber, sorbitan fatty acid ester, film-forming agents, etc.

The cosmetic preparation according to the invention may, apart from the ingredients mentioned for the combination effect, contain other active ingredients and cosmetic auxiliaries and carrier substances.

The cosmetic active ingredients include, for instance, emulsifiers, inorganic and organic sunscreens, further scavengers, moisturisers, further vitamins, enzymes, further plant-based active ingredients, polymers, melanine, antioxidants, anti-inflammatory natural active ingredients, fluorocarbons charged with oxygen or oxygen-carrying asymmetric lamellar aggregates pursuant to WO 94/00109; magnetic single crystals with a great coercive force pursuant to WO 95/03061, e.g. example 2 or 3 and WO 98/44895 e.g. example 1C; kaolin and kaolin modified with SiO₂ pursuant to WO 94/17588.

Glycerine, butyleneglycol, propyleneglycol and mixtures thereof may be preferably used as moisturizers.

Another additive for the cosmetic according to the invention is a plant-based active ingredient preparation with a high radical protection factor as described in WO 99/66881.

Normally, a plurality of compounds may be used as emollients, such as stearyl alcohol, glycerylmonoricinoleate, glycerylmonostearate, propan-1,2-diol, butane-1,3-diol, cetyl alcohol, isopropyliso stearate, stearic acid, isobutyl palmitate, oleyl alcohol, isopropyl laureate, decyl oleate, octadecan-2-ol, isocetyl alcohol, cetyl palmitate, silicone oils such as dimethyl polysiloxane, isopropyl myristate, isopropyl palmitate, polyethylene glycol, lanolin, cocoa butter, vegetable oils such as corn oil, cotton seed oil, olive oil, mineral oils, butyl myristate, palmitic acid, etc.

The mentioned extract of the bark of Quebracho blanco is enzymatically hydrolysed after extraction and contains at least 90 percent by weight proanthocyanidine oligomers and not more than 10 percent by weight gallic acid. Together with a silk worm extract, which is obtained by extraction containing the peptide cecropine, amino acids and a vitamin mixture, both in a non-ionic, cationic or anionic hydro-gel or mixture of hydro-gels constitute a preferred component of the formulation according to the invention, optionally available in microcapsules. The extract mixture is known from WO 99/66881 and is used in the present invention, for instance as an active ingredient complex pursuant to example 1 or 2 of WO 99/66881.

Furthermore, anti-oxidants may be added, such as folic acid and its derivatives, flavones or flavonoides; amino acids, such as histidine, glycine, tyrosine, tryptophane and derivatives thereof; carotinoides and carotenes, such as α-carotene, β-carotene; uric acid and derivatives thereof; α-hydroxy acids such as citric acid, lactic acid, malic acid; stilbenes and their derivatives; as well as pomegranate extracts.

Furthermore, it is advantageous to use water-soluble and/or oil-soluble UVA or UVB filters or both which correspond to the compositions according to the invention. Advantageous oil-soluble UVB filters include 4-aminobenzoic acid derivatives such as the 4-(dimethylamino)-benzoic acid-(2-ethylhexyl) ester; esters of the cinnamic acid such as the 4-methoxy cinnamic acid(2-ethylhexyl)ester, benzophenon derivatives such as 2-hydroxy-4-methoxybenzophenone; 3-benzylidene camphor derivatives such as benzylidene camphor.

Preferred oil-soluble UV filters are Benzophenone-3, Butyl-methoxybenzoyl Methanes, Octyl Methoxy Cinnamates, Octyl Salicylates, 4-Methylbenzylidene Camphor, Homosalate and Octyl dimethyl PABA.

Water-soluble UVB filters are, e.g. sulphonic acid derivatives of benzophenone or of 3-benzylidene camphor or salts, such as the Na or K salt of the 2-phenylbenzimidazole-5-sulphonic acid.

The UVA filters include dibenzoylmethane derivatives, such as 1-phenyl-4-(4′-isopropylphenyl)propane-1-3-dion.

Preferred sunscreen filters are inorganic pigments based on metal oxides, such as TiO₂, SiO₂, ZnO, Fe₂O₃, ZrO₂, MnO, Al₂O₃, which can also be used as a mixture.

Particularly preferred as inorganic pigments are agglomerated substrates of TiO₂ and/or ZnO containing spherical and porous SiO₂ particles, the SiO₂ particles having a particle size in the range of 0.05 μm to 1.5 μm and, apart from the SiO₂ particles, other inorganic particle-shaped substances with a spherical structure are present, the spherical SiO₂ particles forming with the other inorganic substances defined agglomerates with a particle size in the range of 0.06 μm to 5 μm.

Enzymes that are preferably used are enzymes of a cosmetic base complex consisting of an aqueous gel base and contained therein an encapsulated extract of an aqueous extraction of the pineapple fruit and the residue of an aqueous extraction of yogurt, the extractions having been made in each case within a temperature range of 10 to 30° C., the ratio pineapple extract to yogurt extract residue being in the range of 20:80 to 80:20 and the complex having a percentage of the enzyme Bromelin in the range of 0.1 to 1 percent by weight.

Cosmetic compositions with the active ingredient preparation according to the invention may be provided as O/W (oil-in-water) or W/O (water-in-oil) emulsions. Suitable emulsifiers for O/W emulsions are for instance addition products of 2-30 mol ethylene oxide to linear C₈-C₂₂ fatty alcohols, to C₁₂-C₂₂-fatty acids and to C₈-C₁₅-alkyl phenols; C₁₂-C₂₂-fatty acid monoester and diester of addition products of 1-30 mol ethylene oxide to glycerine.

Suitable emulsifiers for W/O emulsions are for instance addition products of 2-15 mol ethylene oxide to castor oil; esters of C₁₂-C₂₂-fatty acids and glycerine, polyglycerine, pentaerythritol, sugar alcohols (e.g. sorbit), polyglucosides (e.g. cellulose); polyalkylene glycols; wool wax alcohols; co-polymers of polysiloxane-polyalkylpolyether.

The oils that are used for the invention may be common cosmetic oils, such as a mineral oil; hydrated polyisobutylene; synthetic squalane or squalane from natural products; cosmetic esters or ethers that may be branched or unbranched, saturated or unsaturated; vegetable oils; or mixtures of two or several of them.

Particularly suited oils are for instance Hydrogenated Polyisobutylene, Polyisoprene, Squalanes, Tridecyltrimellitate, Trimethylpropane-triisostearate, Isodecylcitrate, Neopentylglycoldiheptanoate, PPG-15-Stearylether and vegetable oils, such as calendula oil, jojoba oil, avocado oil, macadamia nut oil, castor oil, wheat germ oil, grape seed oil, kukui nut oil, thistle oil, evening primrose oil, safflower oil or a mixture of several of them.

The cosmetic composition according to the invention may also be provided as a gel. Suited gel-forming agents include Carbomer, Xanthane Gum, Carrageenan, Arabine, Guar Gum, Agaragar, alginates and tyloses, carboxymethylcellulose, hydroxyethylcellulose, quaternized cellulose, quaternized guar, certain polyacrylates, polyvinyl alcohol, polyvinylpyrrolidon, montmorillonite.

The addition of pigments, pigment mixtures or powder with a pigment-like effect to the preparation according to the invention is possible. Such pigments include also pigments with a gloss effect or pigments, such as iron oxides, natural aluminium silicates, such as ochre, titanium oxide, mica, kaolin, manganese-containing clays, such as umbra and red bole, calcium carbonate, talc, mica-titanium oxide, mica-titanium iron oxide, bismuth-oxychloride, nylon pearls, ceramic pearls, expanded and non-expanded synthetic polymer powders, pulverised natural organic compounds, such as ground solid algae, ground plant parts, encapsulated and un-encapsulated cereal starches as well as mica-titanium oxide-organic dye.

The invention relates also to the use of the preparation containing vitamin A described above as a long-term stimulant with an anti-wrinkle effect and scavenging properties.

The cosmetic preparation according to the invention may be used, e.g. in sun creams, sun gels, after-sun products, day creams, night creams, masks, body lotions, cleansing milk, makeups, lip sticks, body powder, eye cosmetics, hair masks, hair rinses, hair shampoos, shower gels, shower oils, bath oils. These products are produced in a manner known to the skilled in this field.

Comparative tests were made with the individual ingredients of the different forms of application of the retinyl derivatives as described in claim 1 a) to d), i. e. the combination of the components a), b) and c) and the combination of the components a), b), c) and d), in each case with the same concentration of the retinyl derivatives as described in example 1. During these tests, the radical protection factor was measured in certain intervals after the application of the preparations on homogeneous skin areas.

The radical protection factor (RPF) determines the activity in respect of the bonding of free radicals by an antioxidant to a test substance. This test substance consists of a very reactive, semi-stable radical that reacts with all known antioxidants. The RPF is measured in such a way that the signal amplitude of the test radical is measured by means of electron spin resonance (ESR/EPR) before and after being mixed with the antioxidant to be determined (here: retinol and/or retinyl derivative and/or other additives with an antioxidant effect) and the RPF is calculated therefrom.

The exact measuring procedure is described by Herrling, Groth, Fuchs and Zastrow in Conference Materials “Modern Challenges To The Cosmetic Formulation” 5.5-7-5.97, Dusseldorf, pages 150-155, Publishing House for the Chemical Industry, 1997. With this procedure, starting from the known concentration of the test radical or the number of its free radicals (radicals per ml), a signal amplitude S₁ is measured by means of an ESR spectrometer. The test radical, just as the antioxidant (here: retinyl derivative, see above) is dissolved in a water/alcohol solution. Then, the signal amplitude S₂ of the antioxidant is measured. The normalised difference between the two signal amplitudes is the reduction factor RF.

RF=(S₁−S₂)/S₁

The result of the test radical reduction RC×RF is normalised to the volume of the product input PI (mg/ml). The radical protection factor is calculated on the basis of the following equation:

$$R P F = \frac{R C [ radicals / ml ] \times R F}{P I [ mg \text{/} ml ]}$$

The result is RPF=N×10¹⁴ [radicals per mg] with N being a positive real figure. The RPF is normally shown without the addition “×10¹⁴” so that a plain smooth figure is obtained.

An RPF<15 does not offer any more significant protection against free radicals.

An RPF of 25 resulted for the combination a) to c) after 4 hours, and for the combination a) to d) an RPF of 67 after 4 hours. After 7 hours, the RPF of the combination a) to d) was 20, and the RPF of the combination a) to c) was 9. This shows a significant prolongation of the duration of action of a combination of different forms of application of retinol and/or of its derivatives on the skin by the addition of a special plant extract. Similar results as with the prunus extract were obtained with a mixture of prunus and acerola extract. Such a mixture is, for instance, available from Greentech S. A., St. Beauzire, France, under the name ROSAMINE.

Below, the invention is to be explained in greater detail by means of examples. All figures are given in percent by weight, unless stated otherwise.

### EXAMPLE 1

### Lotion

a) Content of Vitamin A Active Ingredients:

| | |
|---|---|
| active ingredient A: | 0.5% retinyl palmitate |
| active ingredient B: | 5.0% retinyl palmitate encapsulated in |
| | liposomes (Phoslipon ®) |
| active ingredient C: | 1.0% micro-capsules with 1 percent by |
| | weight pure retinal |
| active ingredient D: | 3.0% extract of the Spanish cherry (Prunus |
| | cerasus) with 0.015% retinyl palmitate |

b) Composition of the Lotion According to Phases

| | | |
|---|---|---|
| Phase A | | |
| Water | ad | 100 |
| Glycerine | | 2 |
| Phase B | | |
| Cetyl Alcohol | | 5 |
| Cetearyl Alcohol | | 5 |
| PEG 40 Stearate | | 1 |
| Phase C | | |
| Vitamin E | | 0.5 |
| active ingredients A, B, C and D | | |
| see above | | |
| Perfume | | 0.5 |
| Preservative | | 0.1 |
| c) procedure | | |

Phases A and B were mixed separately and heated to 75° C. Phase B was added by stirring to phase A and was homogenized for 15 minutes. Thereafter, the mixture was cooled down by stirring to about 32° C., and phase C was added by slowly stirring it until reaching homogeneity.

### EXAMPLE 2

### Anti-Wrinkle Cream

a) Content of Vitamin A Active Ingredients:

| | |
|---|---|
| active ingredient A: | 0.01% retinyl palmitate |
| active ingredient B: | 10.0% retinyl palmitate encapsulated in |
| | liposomes |
| active ingredient C: | 0.1% micro-capsules with 1 percent by |
| | weight pure retinal |
| active ingredient D: | 10.0% extract of the Spanish cherry |
| | (Prunus cerasus) in a mixture with acerola |
| | extract with together 0.02% retinyl palmi- |
| | tate |

b) Composition of the Lotion According to Phases

| | | |
|---|---|---|
| Phase A | | |
| Water | ad | 100 |
| Glycerine | | 3 |
| Cross Polymer | | 0.2 |
| Phase B | | |
| Ssteareth 21 | | 1.8 |
| Ssteareth 2 | | 1.1 |
| Ccetyl Aalcohol | | 2.5 |
| Phase C | | |
| TEA | | 0.2 |
| Phase D | | |
| Ssilicone | | 3 |
| Ppreservative | | 0.8 |
| Pperfume oil | | 0.5 |
| Phase E | | |
| active ingredients A, B, C and D | | |
| see above | | |
| c) procedure | | |

Phases A and B are mixed separately and heated to 75° C. Phase B is added by stirring to phase A and was homogenized for 15 minutes. Thereafter, the mixture is cooled down by stirring to about 32° C., and one after the other the phases C, D and E are added by slowly stirring it until reaching homogeneity.

### EXAMPLES 3-6

Anti-wrinkle creams with the composition of the lotion pursuant to example 2b) are made using the same procedure. The contents of the vitamin A active ingredients is as follows (in percent by weight, calculated with reference to the entire preparation):

TABLE 1

| active ingredient | Example 3 | Example 4 | Example 5 | Example 6 |
|---|---|---|---|---|
| A | | | | |
| retinyl ascorbate | 0.8 | — | 0.6 | 0.8 |
| retinyl palmitate | — | 0.1 | 0.05 | — |
| B | | | | |
| retinyl ascorbate | — | — | 6.0 | — |
| retinyl palmitate | 8.0 | 1.1 | — | 8.0 |
| C | | | | |
| Retinol | 2.4 | 4.2 | 0.08 | — |
| D | | | | |
| Quebracho extract | 0.3 | — | — | 0.3 |
| pineapple extract | — | 0.9 | — | — |
| silk worm extract | — | — | 3.3 | — |

Here, example 6, which does not contain the active ingredient C (encapsulated retinol), is a comparative example and can be compared to example 3. Retinyl derivatives of the active ingredient D are in example 1 retinol, in examples 2, 3 and 6 retinyl palmitate, in example 4 retinyl ascorbate and in example 5 retinol+retinyl palmitate.

### EXAMPLE 7

Tests were made for proving the anti-wrinkle effect. The test was made with 21 male/female test persons aged between 42 and 58. The micro-relief of areas of the skin of the face (area of the eyes, corners of the mouth, area of the nose) was taken by means of a silicone mass, the mass was hardened and the negative relief that was obtained was measured electro-optically as to the depth and the number of the wrinkles. Immediately following the taking of the micro-relief, a cream was applied for the first time to the faces of the test persons. Thereafter, the application was repeated twice daily.

Group 1: 12 test persons were given the cream of example 3;

Group 2: 6 test persons were given the cream of example 6;

Group 3: 3 test persons were given a cream that consisted solely of the base formulation without active ingredients (placebo).

Control measurements by taking the micro-relief of the same skin areas of the individual test persons were made on day 14 and on day 28 after the first measurement. During that period, all test persons were not exposed to any special strain by sun radiation. The values that were determined are statistical mean values over a selected range of the micro-relief.

TABLE 2

| | Number of test persons | | |
|---|---|---|---|
| Improvement of wrinkle depth | group 1 | group 2 | group 3 |
| after 14 days | | | |
| by 10 to 30% | 9 | 4 | 1 |
| by 30 to 50% | 2 | — | — |
| after 28 days | | | |
| by 30 to 50% | 3 | 3 | — |
| by 50 to 60% | 6 | 1 | — |
| by 60 to 70% | 3 | — | — |

Group 1 shows a very good reduction of the wrinkle depth after 28 days in 75% of the test persons. In comparison to Group 2, to which a combination of different vitamin A derivatives was also applied, the composition according to the invention of example 3 shows another significant improvement which was not to be readily expected from the increased retinol content in comparison with example 6, but rather points to a still not clearly understood synergism.

1. Cosmetic preparation containing vitamin A which comprises
a) 0.001 to 1.0 percent by weight of a retinyl derivative, calculated with reference to the weight of the cosmetic preparation and selected from among retinyl palmitate, retinyl ascorbate and mixtures thereof; and
b) 0.01 to 10 percent by weight of a retinyl derivative, selected from among retinyl palmitate, retinyl ascorbate and mixtures thereof and encapsulated in phospholipid-containing liposomes with a particle size of the liposomes in the range of 150 to 800 nm, the content of retinyl derivatives being calculated with reference to the weight of the liposomes; and
c) 0.01 to 5 percent by weight of micro-capsules with pure retinol the particle size of the capsules being in the range of 40 to 100 nm and which contain pure retinol in the range of 0.5 to 3 percent by weight, calculated with reference to the weight of the filled capsules; and
d) 0.1 to 20 percent by weight, calculated with reference to the weight of the cosmetic preparation, of an extract, selected from among the aqueous fruit extracts cherry extract (Prunus cerasus), acerola extract (Malpighia punicifolia), pineapple extract, hydrolized bark extract of Quebracho blanco, silkworm extract and mixtures of these extracts, with the content of the extract of at least one retinol compound, selected from among retinol, retinyl palmitate, retinyl ascorbate and mixtures thereof, being in the range of 0.01 to 5 percent by weight, this figure being calculated with reference to the dry weight of the extract; and
e) a rest, up to 100 percent by weight, of cosmetic auxiliaries, carrier substances and mixtures thereof.

2. Preparation according to claim 1 wherein the fruit extract is a mixture of a cherry extract with an acerola extract.

3. Preparation according to claim 1 wherein the cherry is a Spanish cherry.

4. Preparation according to claim 2 wherein the fruit extract mixture is encapsulated in capsules with a particle size of 30 to 100 μm.

5. Preparation according to claim 4 wherein the retinyl palmitate which is contained in the micro-capsules is provided as a mixture together with at least another vitamin, selected from among a vitamin E derivative, vitamin C, vitamin F and mixtures thereof, and carotenoides.

6. Preparation according to claim 1 wherein the retinyl derivative that is encapsulated in liposomes is retinyl palmitate.

7. Preparation according to one of the claims 1 to 6 which comprises other active ingredients, selected from among sun screens, scavengers, enzymes, active ingredient extracts of plant-based substances, oxygen-carrying fluorocarbons, magnetic single crystals with a high coercive force, kaolin modified with SiO₂ and mixtures thereof.

8. Preparation according to claim 1 which comprises in addition a cosmetic base complex consisting of an aqueous gel base and contained therein an encapsulated extract of an aqueous extraction of the pineapple fruit and the residue of an aqueous extraction of yogurt the extractions in each case having been made in the temperature range of 10 to 30° C., the ratio pineapple extract to yogurt extract residue being in the range of 20:80 to 80:20 and the complex having a percentage of the enzyme Bromelin in the range of 0.1 to 1 percent by weight.

9. Use of a cosmetic preparation containing vitamin A according to one of the claims 1 to 8 as a long-term stimulant with an anti-wrinkle effect and scavenging properties.
